(19) United States
(12) Patent Application Publication
(10) Pub. No.: US 2020/0360001 A1
(43) Pub. Date: Nov. 19, 2020

(54) Frustoconical Hemostatic Sealing Elements

(71) Applicant: 4Tech Inc., Waltham, MA (US)

(72) Inventors: Evin Donnelly, Galway (IE); Patrick Griffin, Galway (IE)

(73) Assignee: 4Tech Inc., Waltham, MA (US)

(21) Appl. No.: 16/635,993

(22) PCT Filed: Feb. 7, 2019
(86) PCT No.: PCT/US2019/016946

§ 371 Date: Jan. 31, 2020

Related U.S. Application Data
(60) Provisional application No. 62/628,457, filed on Feb. 9, 2018.

(51) Int. Cl. A61B 17/00

(52) U.S. Cl. CPC A61B 17/0057; A61B 2017/00884; A61B 2017/00898; A61B 2017/00676; A61B 2017/00592; A61B 2017/00597; A61B 2017/00623; A61B 2017/00867

(57) ABSTRACT

A hemostatic tissue anchor (120) is provided that includes an anchor portion (130) supported at a distal end (192) of a generally elongate anchor shaft (132). A hemostatic sealing element (122) is coupled to and surrounds at least an axial portion of the anchor shaft (132), is configured to be disposed at least partially within a cardiac tissue wall (160) at a target site, and includes a self-expanding frame (124) attached to a sealing membrane (126). The hemostatic sealing element (122) includes an expandable portion (128) that assumes an expanded frustoconical configuration (138) that is defined by the self-expanding frame (124) and the sealing membrane (126), and acts as a hemostatic seal of an opening through the cardiac tissue wall (160), through which opening the anchor shaft (132) is disposed. Other embodiments are also described.

### CROSS-REFERENCE TO RELATED APPLICATIONS

The present application claims priority from U.S. Provisional Application 62/628,457, filed Feb. 9, 2018, which is assigned to the assignee of the present application and incorporated herein by reference.

### FIELD OF THE APPLICATION

The present invention relates generally to tissue anchors, and specifically to tissue anchors for implantation at cardiac sites.

### BACKGROUND OF THE APPLICATION

Tissue anchors are used for anchoring elements, such as pacemaker electrode leads or sutures, to tissue, such as bone or soft tissue. PCT Publication WO 2016/087934 to Gilmore et al., which is incorporated in its entirety herein by reference, describes a tissue anchor that includes a shaft, a tissue-coupling element, and a flexible elongate tension member. The tissue-coupling element includes a wire, which is shaped as an open loop coil having, in some applications, more than one coil revolution when the tissue anchor is unconstrained, i.e., expanded from a linear state to a coiled state. The tension member includes a distal portion, that is fixed to a site on the open loop coil, a proximal portion, which has a longitudinal segment that runs alongside at least a portion of the shaft, and a crossing portion, which (i) is disposed between the distal and the proximal portions along the tension member, and (ii) crosses at least a portion of the open loop when the tissue anchor is expanded. The tissue anchor is configured to allow relative axial motion between the at least a portion of the shaft and the longitudinal segment of the proximal portion of the tension member when the tissue anchor is expanded. For some applications, the shaft comprises a sealing element, which is configured to form a blood-tight seal between a portion of the shaft inside the heart chamber and the wall of the heart.

U.S. Pat. No. 8,758,402 to Jenson et al. describes methods and devices for closing and/or sealing an opening in a vessel wall and/or an adjacent tissue tract. The '402 Patent describes a device for delivering and deploying an anchor, plug, suture, and/or locking element adjacent to the opening in the vessel wall and/or tissue tract.

US Patent Application Publication 2012/0172928 to Eidenschink et al. describes a device for sealing a puncture opening that may include a base frame having a delivery configuration, the base frame being retracted to have a relatively smaller overall profile, and a deployed configuration, the base frame being extended to have a relatively larger overall profile. The base frame is sized to engage an interior surface of the blood vessel wall in the deployed configuration. A sealing section is coupled to the base frame, the sealing section having an initial configuration, the sealing section permitting fluid flow, and a barrier configuration, the sealing section preventing fluid flow. The sealing section in the barrier configuration is sized to block fluid flow through the puncture opening when the base frame is in the deployed configuration.

### SUMMARY OF THE APPLICATION

Embodiments of the present invention provide a hemostatic tissue anchor deliverable within a hollow delivery shaft to a target site. The hemostatic tissue anchor is configured to be anchored to a cardiac tissue wall at the target site. The hemostatic tissue anchor comprises an anchor portion supported at a distal end of a generally elongate anchor shaft. The anchor portion is configured to expand from a first generally elongate configuration within the hollow delivery shaft during delivery of the hemostatic tissue anchor, to a second expanded configuration, upon release from the hollow delivery shaft, such that the anchor portion in the second expanded configuration can be drawn tightly against the cardiac tissue wall at the target site when a tensile force is applied to the anchor portion.

The hemostatic tissue anchor further comprises a hemostatic sealing element, which is coupled to and surrounds at least an axial portion of the elongate anchor shaft. The hemostatic sealing element is configured to be disposed at least partially within the cardiac tissue wall at the target site. The hemostatic sealing element typically comprises a self-expanding frame attached to a sealing membrane. The hemostatic sealing element comprises an expandable portion that assumes a collapsed configuration within the hollow delivery shaft during delivery of the hemostatic tissue anchor, and, upon release from the hollow delivery shaft at least partially within the cardiac tissue wall, an expanded frustoconical configuration, the expanded frustoconical configuration defined by the self-expanding frame and the sealing membrane. Once the expandable portion of the hemostatic sealing element is implanted at least partially within the cardiac tissue wall at the target site, the expanded frustoconical configuration of the hemostatic sealing element acts as a hemostatic seal of an opening through the cardiac tissue wall, through which opening the elongate anchor shaft is disposed.

For some applications, the expanded frustoconical configuration widens in the distal direction, while for other applications, the expanded frustoconical configuration widens in the proximal direction.

For some applications, the self-expanding frame is embedded in the sealing membrane.

For some applications, the sealing membrane is electrospun.

For some applications, the sealing membrane is dip-coated or laminated onto the self-expanding frame.

For some applications, the sealing membrane is woven.

For some applications, the sealing membrane includes a fabric.

For some applications, the sealing membrane includes a hygroscopic polymer, which, when exposed to fluid, absorbs moisture and expands.

For some applications, the self-expanding frame of the expanded frustoconical configuration is shaped so as define a plurality of distally- or proximally-extending crowns.

For any of the applications described above, the self-expanding frame may include metal. For some applications, the self-expanding metal frame includes metal wires braided into the sealing membrane.

For any of the applications described above, the self-expanding frame may include a hygroscopic polymer, which, when exposed to fluid, absorbs moisture and expands, thereby driving the expandable portion to assume the expanded frustoconical configuration.

For any of the applications described above, the expanded frustoconical configuration may have a greatest diameter that is greater than an outer diameter of the hollow delivery shaft.

For any of the applications described above, the elongate anchor shaft may include an anchor head that defines the distal end of the anchor shaft, the expanded frustoconical configuration may have a distal end that is disposed proximal to the distal end of the anchor head, and the hemostatic sealing element may be configured to be disposed entirely within the cardiac tissue wall at the target site.

For any of the applications described above, the elongate anchor shaft may include an anchor head that defines the distal end of the anchor shaft, the expanded frustoconical configuration may have a distal end that is disposed distal to the distal end of the anchor head, and the hemostatic sealing element may be configured to be disposed only partially within the cardiac tissue wall at the target site, with a distal portion of the hemostatic sealing element, including the distal end of the expanded frustoconical configuration, expanded in the pericardial cavity between visceral pericardium and parietal pericardium. For some applications, the hemostatic sealing element is configured such that when the distal portion of the hemostatic sealing element is expanded in the pericardial cavity, the distal portion of the hemostatic sealing element assumes a trumpet-bell shape. For some applications, the sealing membrane has a greater thickness at a first axial location at which the sealing membrane axially overlaps a wire of the anchor portion distal to the distal end of the anchor head than at a second axial location at which the sealing membrane axially overlaps the anchor head, when the hemostatic tissue anchor is constrained within the hollow delivery shaft.

For any of the applications described above, the cardiac tissue wall may be a myocardial tissue wall, and the expandable portion of the hemostatic sealing element may be configured to be implanted at least partially within the myocardial tissue wall. For some applications, the anchor portion is configured to be implanted in the pericardial cavity between visceral pericardium and parietal pericardium, generally alongside and against the parietal pericardium, without penetrating the parietal pericardium.

For any of the applications described above, the anchor portion, when expanded, may define a generally planar structure orthogonal to the elongate anchor shaft.

There is further provided, in accordance with an application of the present invention, a method for anchoring a hemostatic tissue anchor to a cardiac tissue wall at a target site, the method including:

delivering within a hollow delivery shaft, to a cardiac chamber, the hemostatic tissue anchor, the hemostatic tissue anchor including:
an anchor portion supported at a distal end of a generally elongate anchor shaft, the anchor portion configured to expand from a first generally elongate configuration within the hollow delivery shaft during delivery of the hemostatic tissue anchor, to a second expanded configuration, upon release from the hollow delivery shaft, such that the anchor portion in the second expanded configuration can be drawn tightly against the cardiac tissue wall at the target site when a tensile force is applied to the anchor portion, and
a hemostatic sealing element, which (a) is coupled to and surrounds at least an axial portion of the elongate anchor shaft, (b) is configured to be disposed at least partially within the cardiac tissue wall at the target site, and (c) includes a self-expanding frame attached to a sealing membrane;

delivering (a) the anchor portion in an unexpanded generally elongate configuration within the hollow delivery shaft through the cardiac tissue wall from a first side of the wall to a second side of the wall, such that the anchor portion expands on the second side of the cardiac tissue wall, thereby anchoring the tissue anchor to the cardiac tissue wall at the target site, and (b) an expandable portion of the hemostatic sealing element in a collapsed configuration within the hollow delivery shaft; and

releasing the hemostatic sealing element from the hollow delivery shaft at least partially within the cardiac tissue wall at the target tissue site, such that the hemostatic sealing element assumes an expanded frustoconical configuration within the cardiac tissue wall that acts as a hemostatic seal of an opening through the cardiac tissue wall, through which opening the elongate anchor shaft is disposed, the expanded frustoconical configuration defined by the self-expanding frame and the sealing membrane.

For some applications, the expanded frustoconical configuration widens in the distal direction. For other applications, the expanded frustoconical configuration widens in the proximal direction.

For some applications, the self-expanding frame is embedded in the sealing membrane. For some applications, the sealing membrane is electrospun. For some applications, the sealing membrane is dip-coated or laminated onto the self-expanding frame. For some applications, the sealing membrane is woven. For some applications, the sealing membrane includes a fabric.

For some applications, the sealing membrane includes a hygroscopic polymer, which, when exposed to fluid, absorbs moisture and expands. For some applications, the self-expanding frame of the expanded frustoconical configuration is shaped so as define a plurality of distally- or proximally-extending crowns.

For some applications, the self-expanding frame includes metal. For some applications, the self-expanding metal frame includes metal wires braided into the sealing membrane.

For some applications, the self-expanding frame includes a hygroscopic polymer, which, when exposed to fluid, absorbs moisture and expands, thereby driving the expandable portion to assume the expanded frustoconical configuration.

For some applications, the expanded frustoconical configuration has a greatest diameter that is greater than an outer diameter of the hollow delivery shaft.

For some applications, the elongate anchor shaft includes an anchor head that defines the distal end of the anchor shaft, the expanded frustoconical configuration has a distal end that is disposed proximal to the distal end of the anchor head, and releasing the hemostatic sealing element includes releasing the hemostatic sealing element from the hollow delivery shaft entirely within the cardiac tissue wall at the target tissue site.

For some applications, the elongate anchor shaft includes an anchor head that defines the distal end of the anchor shaft, the expanded frustoconical configuration has a distal end that is disposed distal to the distal end of the anchor head, and releasing the hemostatic sealing element includes releasing the hemostatic sealing element from the hollow delivery shaft only partially within the cardiac tissue wall at the target tissue site, with a distal portion of the hemostatic sealing element, including the distal end of the expanded frustoconical configuration, expanded in the pericardial cavity between visceral pericardium and parietal pericardium. For some applications, releasing the distal portion of the hemostatic sealing element in the pericardial cavity causes the distal portion of the hemostatic sealing element to assume a trumpet-bell shape. For some applications, the sealing membrane has a greater thickness at a first axial location at which the sealing membrane axially overlaps a wire of the anchor portion distal to the distal end of the anchor head than at a second axial location at which the sealing membrane axially overlaps the anchor head, when the hemostatic tissue anchor is constrained within the hollow delivery shaft.

For some applications, the cardiac tissue wall is a myocardial tissue wall, and releasing includes releasing the hemostatic sealing element from the hollow delivery shaft within the myocardial tissue wall. For some applications, delivering the anchor portion in the unexpanded generally elongate configuration through the cardiac tissue wall includes delivering the anchor portion through the myocardial tissue wall into the pericardial cavity between visceral pericardium and parietal pericardium, generally alongside and against the parietal pericardium, without penetrating the parietal pericardium.

For some applications, delivering the anchor portion in the unexpanded generally elongate configuration through the cardiac tissue wall includes delivering the anchor portion such that the anchor portion, when expanded, defines a generally planar structure orthogonal to the elongate anchor shaft.

The present invention will be more fully understood from the following detailed description of embodiments thereof, taken together with the drawings, in which:

### BRIEF DESCRIPTION OF THE DRAWINGS

FIGS. 1A-B are schematic illustrations of a hemostatic tissue anchor that is configured to be anchored to a cardiac tissue wall at a target site, in accordance with respective applications of the present invention;

FIGS. 2A-C are schematic illustrations of the deployment of the hemostatic tissue anchor of FIG. 1A, in accordance with an application of the present invention;

FIGS. 3A-B are schematic illustrations of the expanded frustoconical configuration of the hemostatic sealing element of the hemostatic tissue anchor of FIGS. 1A-B, in accordance with respective applications of the present invention;

FIG. 4 is a schematic illustration of another hemostatic sealing element, in accordance with an application of the present invention;

FIG. 5 is a schematic illustration of a portion of another hemostatic tissue anchor, in accordance with an application of the present invention; and

FIGS. 6A-B are schematic illustrations of yet another hemostatic tissue anchor, in accordance with an application of the present invention.

### DETAILED DESCRIPTION OF APPLICATIONS

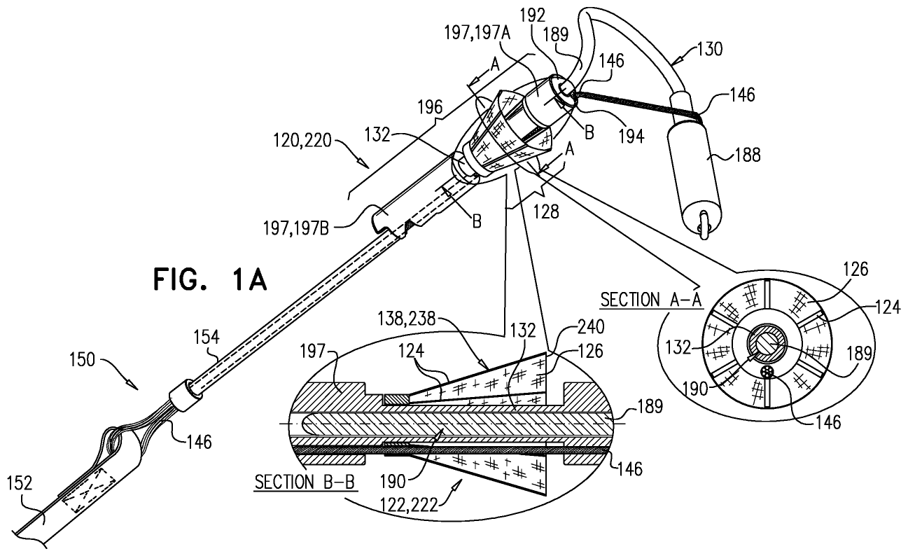

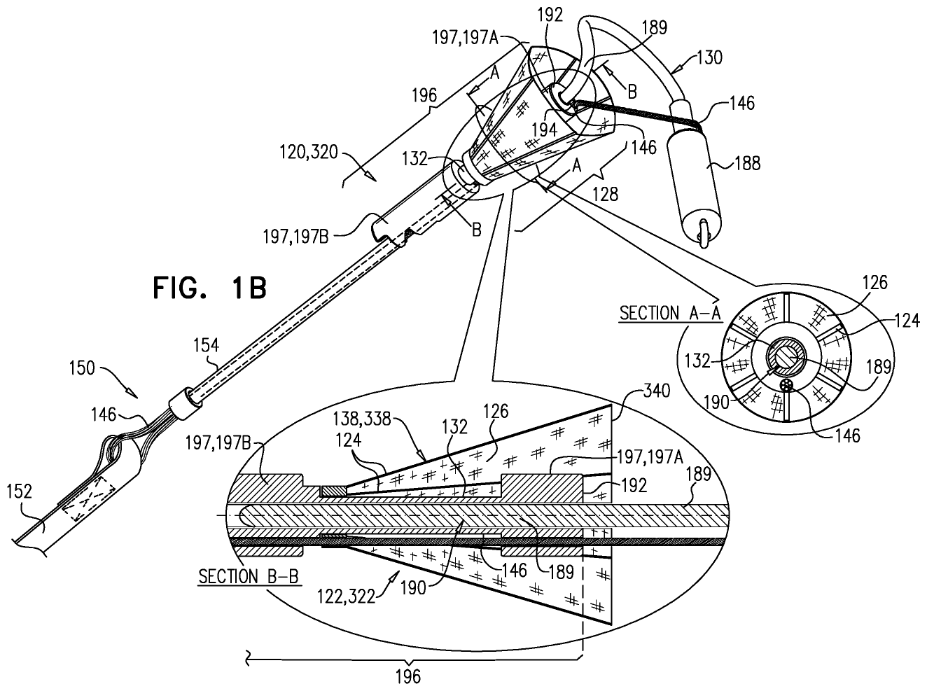

FIGS. 1A-B are schematic illustrations of a hemostatic tissue anchor 120 that is configured to be anchored to a cardiac tissue wall 160 at a target site, in accordance with respective applications of the present invention. FIG. 1A is a schematic illustration of a hemostatic tissue anchor 220, and FIG. 1B is a schematic illustration of a hemostatic tissue anchor 320. Hemostatic tissue anchors 220 and 320 are implementations of hemostatic tissue anchor 120, and are identical other than as described hereinbelow and shown in the figures.

Hemostatic tissue anchor 120 comprises an anchor portion 130, supported at a distal end 192 of a generally elongate anchor shaft 132. FIGS. 1A-B show anchor portion 130 expanded. For some applications, such as shown, elongate anchor shaft 132 comprises an anchor head 196, which may define distal end 192.

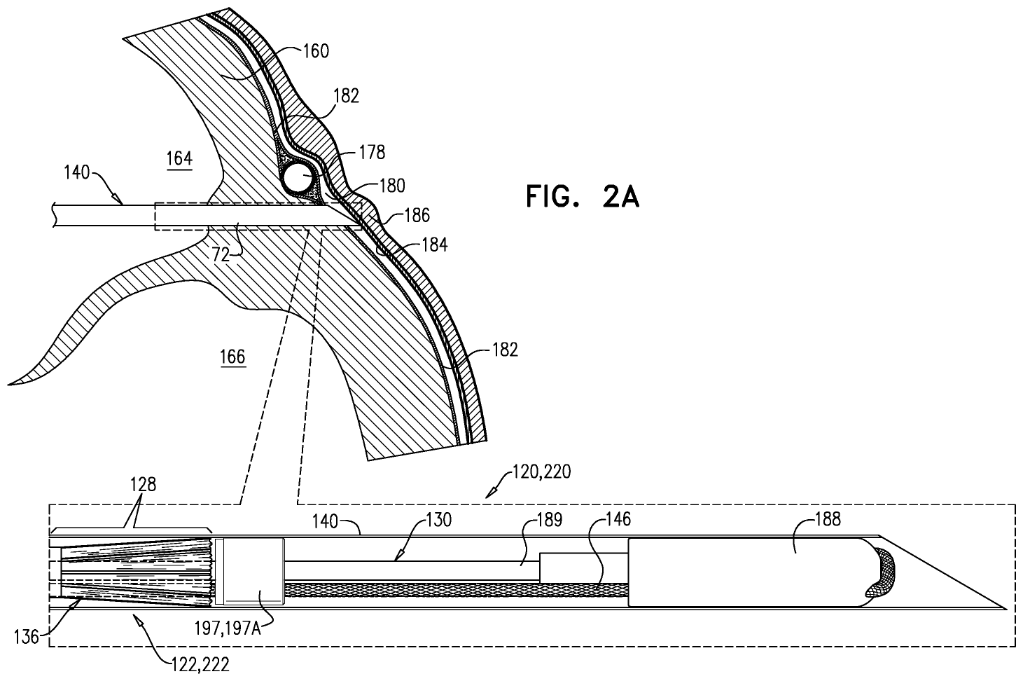

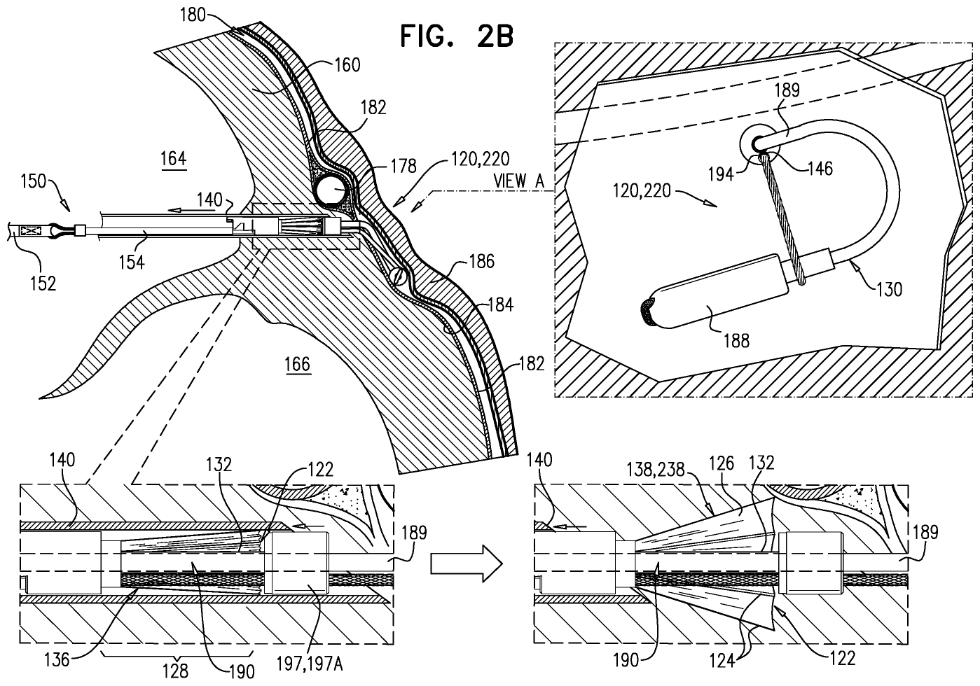

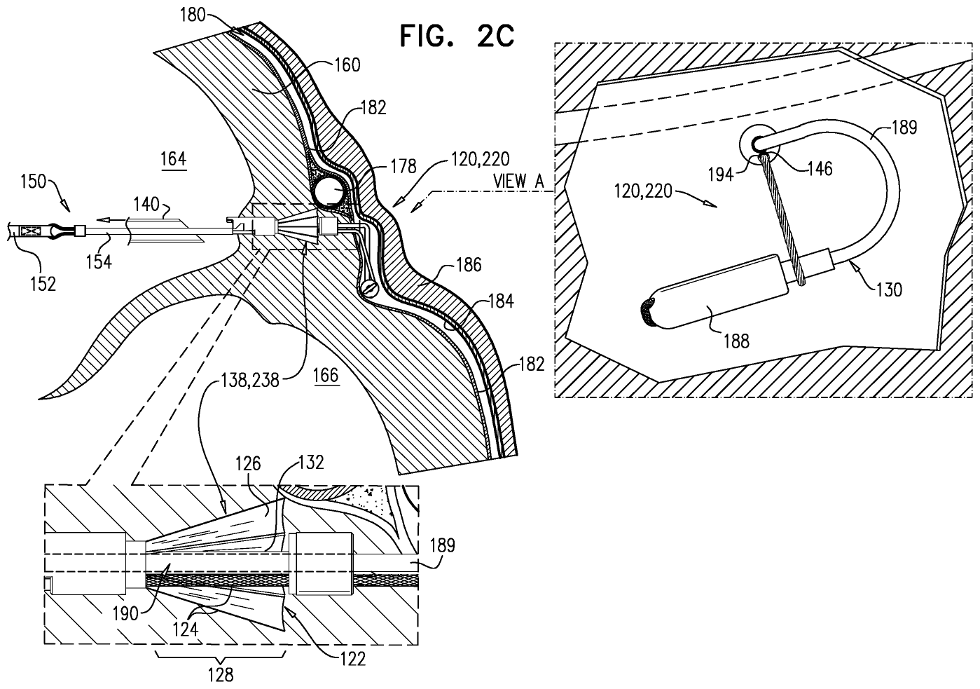

Reference is also made to FIGS. 2A-C, which are schematic illustrations of the deployment of hemostatic tissue anchor 120, in accordance with an application of the present invention. As shown in FIG. 2A, hemostatic tissue anchor 120 is deliverable to the target site through cardiac tissue wall 160 (e.g., a myocardial tissue wall) from a first side of the wall to a second side of the wall, with anchor portion 130 in an unexpanded first generally elongate configuration within a hollow delivery shaft 140. Although FIGS. 2A-C show the deployment of hemostatic tissue anchor 220, described herein with reference to FIG. 1A, the same techniques may be used to deploy hemostatic tissue anchor 320, described herein with reference to FIG. 1B, mutatis mutandis.

As shown in FIG. 2B, anchor portion 130 is further configured, upon deployment from hollow delivery shaft 140, to expand on the second side of the cardiac tissue wall to a second expanded configuration, such that anchor portion 130 in the second expanded configuration can be drawn tightly against the cardiac tissue wall at the target site when a tensile force is applied to anchor portion 130. For some applications, anchor portion 130, once expanded on the second side of the cardiac tissue wall, defines a generally planar structure orthogonal to elongate anchor shaft 132, as shown in FIGS. 1A-B and 2B-C, although it need not be orthogonal.

Hemostatic tissue anchor 120 further comprises a hemostatic sealing element 122, which is coupled to and surrounds at least an axial portion of elongate anchor shaft 132. Hemostatic sealing element 122 is configured to be disposed at least partially within cardiac tissue wall 160 at the target site. In some configurations, such as shown in FIGS. 1A and 2A-C, hemostatic sealing element 122 comprises a hemostatic sealing element 222 that is configured to be disposed entirely within cardiac tissue wall 160 at the target site. In other configurations, such as shown in FIG. 1B, hemostatic sealing element 122 comprises a hemostatic sealing element 322 that is configured to be disposed only partially within cardiac tissue wall 160 at the target site, with a distal portion of hemostatic sealing element 322 expanded on the far side of cardiac tissue wall 160, e.g., in the pericardial cavity 180.

For some applications, hemostatic sealing element 122 comprises a self-expanding frame 124 attached to a sealing membrane 126.

As shown in FIG. 2A, hemostatic sealing element 122 comprises an expandable portion 128 that assumes a collapsed configuration 136 within hollow delivery shaft 140 during delivery of hemostatic tissue anchor 120. As hemostatic tissue anchor 120 is delivered, hollow delivery shaft 140 pushes away cardiac tissue laterally from the longitudinal axis of hemostatic tissue anchor 120.

As shown in FIG. 2B, once hemostatic tissue anchor 120 is properly positioned such that hemostatic sealing element 122 is disposed at least partially within the cardiac tissue, delivery shaft 140 is gradually retracted proximally so as to expose and release hemostatic sealing element 122.

As shown in FIG. 2C, upon release from hollow delivery shaft 140 at least partially within cardiac tissue wall 160, expandable portion 128 of hemostatic sealing element 122 assumes an expanded frustoconical configuration 138 that may widen in the distal direction, as shown. Alternatively, expanded frustoconical configuration 138 widens in the proximal direction, in which case blood flow may drive expansion of hemostatic sealing element 122, i.e., like a parachute catching air. Expanded frustoconical configuration 138 is defined by self-expanding frame 124 and sealing membrane 126. Typically, as hemostatic sealing element 122 is exposed from within hollow delivery shaft 140, the cardiac tissue closes around expanded frustoconical configuration 138, such that expanded frustoconical configuration 138 functions as a hemostatic seal.

Also as shown in FIG. 2C, once expandable portion 128 of hemostatic sealing element 122 is implanted at least partially within cardiac tissue wall 160 at the target site, expanded frustoconical configuration 138 acts as a hemostatic seal of an opening (i.e., incision) through cardiac tissue wall 160 (e.g., within the myocardial tissue wall), through which opening elongate anchor shaft 132 is disposed. Hemostatic sealing element 122, typically along with at least a portion of elongate anchor shaft 132, remains in the opening through cardiac tissue wall 160 upon completion of the implantation of hemostatic tissue anchor 120. Sealing element 122 promotes hemostasis to provide sealing of the opening through cardiac tissue wall 160.

Cardiac tissue wall 160 may be of a right atrium 164 (as shown in FIGS. 2A-C), a right ventricle 166 (configuration not shown), a left atrium (configuration not shown), or a left ventricle (configuration not shown). For some applications, as shown in FIGS. 2A-C, hollow delivery shaft 140 is used to puncture through a first side of myocardial tissue wall 160 and visceral pericardium 182 (which is part of the epicardium), avoiding vasculature such as the right coronary artery (RCA) 178. For some applications, hollow delivery shaft 140 is then further directed into the pericardial cavity 180 between visceral pericardium 182 and parietal pericardium 184, carefully avoiding puncturing parietal pericardium 184 and fibrous pericardium 186. For some applications, anchor portion 130 is configured to be implanted in pericardial cavity 180 between visceral pericardium 182 and parietal pericardium 184, generally alongside and against parietal pericardium 184, without penetrating the parietal pericardium 184.

Once hemostatic tissue anchor 120 has been anchored to myocardial tissue wall 160 at the target site, expanded anchor portion 130 is tightly drawn against the second side of myocardial tissue wall 160 at the target site by applying a tensile force, such as using tether 152, described hereinbelow, to anchor portion 130 to myocardial tissue wall 160. Application of the tensile force partially compresses expanded anchor portion 130. For applications in which expanded frustoconical configuration 138 widens in the distal direction, the tapered surface of expanded frustoconical configuration 138 provides an atraumatic interface between frustoconical configuration 138 and surrounding cardiac tissue, in particular, during the application of the tensile forces.

Although in FIGS. 2A-C hemostatic tissue anchor 120 is shown deployed through a myocardial tissue wall, hemostatic tissue anchor 120 may also be deployed through other cardiac tissue walls, such as the interatrial septum, either at or not at the fossa ovalis, or through other non-cardiac tissue walls. Indeed, the tissue anchors described herein may be deployed in any number of bodily locations where it is desired to anchor into or behind tissue for purposes of moving such tissue relative to adjacent tissue.

For some applications, self-expanding frame 124 comprises metal. For example, self-expanding metal frame 124 may comprise a superelastic allay, such as Nitinol, or other springy metal, such as steel. Alternatively, self-expanding metal frame 124 may comprise a bioabsorbable metal, such as a magnesium alloy, in order to allow bioabsorption of the frame over time once hemostasis has been achieved and wound has healed. For some applications, sealing membrane 126 comprises a hygroscopic polymer, which, when exposed to fluid (e.g., blood and/or pericardial fluid), absorbs moisture and expands (i.e., swells).

For other applications, self-expanding frame 124 comprises a hygroscopic polymer, which, when exposed to fluid (e.g., blood and/or pericardial fluid), absorbs moisture and expands (i.e., swells), thereby driving expandable portion 128 to assume expanded frustoconical configuration 138, in order to seal the channel through the cardiac wall. In applications in which self-expanding frame 124 comprises a hygroscopic polymer, no sealing membrane may be needed. In applications in which sealing membrane 126 is provided, the hygroscopic polymer frame may be dispensed, printed, or stitched onto sealing membrane 126, and/or may be arranged in a stent pattern on sealing membrane 126. For some applications, the hygroscopic polymer frame 124 is impregnated into sealing membrane 126. For example, sealing membrane 126 may be porous, e.g., may comprise an electrospun polymer matrix or open cell polymer foam soaked in a hydrogel then dried out for delivery; upon rehydration in vivo the hydrogel swells, expanding the matrix.

For some applications, such as shown in FIG. 2B, expanded frustoconical configuration 138 has a greatest diameter D1 that is greater than an outer diameter D2 of hollow delivery shaft 140; for example, greatest diameter D1 of expanded frustoconical configuration 138 may equal at least 105% of the outer diameter D2 of hollow delivery shaft 140. Alternatively or additionally, for some applications, greatest diameter D1 of expanded frustoconical configuration 138 equals at least 100% of an outer diameter D3 of elongate anchor shaft 132.

Reference is again made to FIGS. 1A and 1B. For some applications, such as shown in FIG. 1A, expanded frustoconical configuration 138 of hemostatic sealing element 222 comprises an expanded frustoconical configuration 238 that has a distal end 240 that is disposed proximal to distal end 192 of anchor head 196 (and thus proximal to distal collar 197A in configurations in which distal collar 197A is provided). In these applications, hemostatic sealing element 222 is typically configured to be disposed entirely within cardiac tissue wall 160 at the target site, such as shown in FIG. 2C.

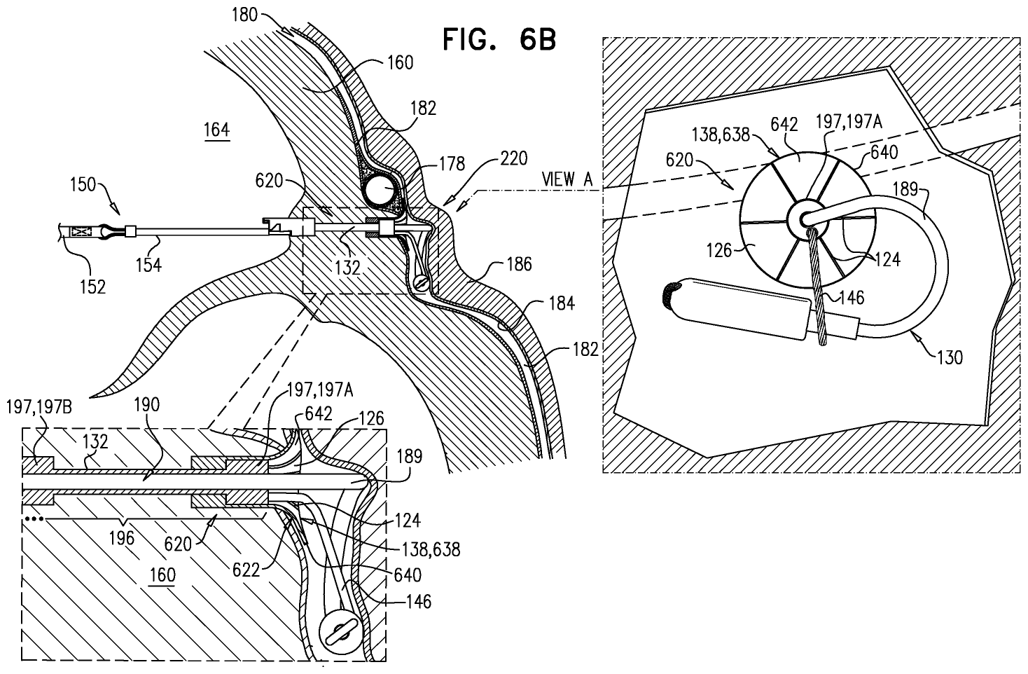

For other applications, such as shown in FIG. 1B, expanded frustoconical configuration 138 of hemostatic sealing element 322 comprises an expanded frustoconical configuration 338 that has a distal end 340 that is disposed distal to distal end 192 of anchor head 196 (and thus distal to distal collar 197A in configurations in which distal collar 197A is provided). Distal end 340 may touch, or come near to, anchor portion 130. In these applications, hemostatic sealing element 322 is typically configured to be disposed only partially within cardiac tissue wall 160 at the target site, with a distal portion of hemostatic sealing element 322, including distal end 340, expanded on the far side of cardiac tissue wall 160, e.g., in the pericardial cavity 180. For some applications, self-expanding frame 124 and sealing membrane 126 are shaped and configured to maintain the strictly conical shape of the distal portion of expanded frustoconical configuration 338 when expanded in the pericardial cavity 180, as shown in FIG. 1B. Alternatively, self-expanding frame 124 and sealing membrane 126 are shaped and configured to allow expanded frustoconical configuration 338 to assume a trumpet-bell shape, such as described hereinbelow with reference to FIG. 6B.

For some applications, expanded anchor portion 130 has less than one turn, as shown in the figures, while for other applications, expanded anchor portion 130 has one turn (configuration not shown) or more than one turn (configuration not shown, but, for example, may be as shown in FIGS. 5B-D, 6A-B, 7A-B, 9A-G, and/or 91 of above-mentioned PCT Publication WO 2016/087934).

Reference is still made to FIGS. 1A-B and 2A-C. For some applications, anchor portion 130 comprises a tip 188, which is fixed to a distal end of a wire 189 of anchor portion 130. Tip 188, at a widest longitudinal site along tip 188, has a greatest outer cross-sectional area that equals at least 150% (e.g., at least 200%, or at least 300%) of an average cross-sectional area of wire 189. (The cross-sectional area of tip 188 is measured perpendicular to a central longitudinal axis of tip 188. Similarly, the cross-sectional area of wire 189 is measured perpendicular to a central longitudinal axis of the wire, and is not a cross-sectional area of anchor portion 130.) Wire 189 may be solid or hollow (i.e., tubular). (Optionally, wire 189, in addition to the portion that defines anchor portion 130, also defines a wire-shaft portion 190 that is inserted into anchor shaft 132 and/or anchor head 196 if provided.)

For some applications, hollow delivery shaft 140 comprises a hollow needle and a sharp distal end of the hollow needle extends distally beyond the distal end of distal tip 188, such that distal tip 188 is disposed within the hollow needle, such as shown in FIG. 2A. Alternatively, hollow delivery shaft 140 does not comprise a sharp distal tip, and instead distal tip 188 is shaped so as to define a sharp dilator tip (configuration not shown). Distal tip 188 is disposed such that a proximal end of the distal tip 188 is flush with a distal end of hollow delivery shaft 140, and thus serves as a distal cap of hollow delivery shaft 140. For some of these applications, hollow delivery shaft 140 has an outer cross-sectional area which equals between 90% and 110% (e.g., 100%) of the greatest outer cross-sectional area of distal tip 188. This latter configuration may allow the use of a lower profile hollow delivery shaft 140 than in the former configuration, because the bore of the shaft does need to accommodate the relative wide distal tip 188. Such a lower profile may reduce the wound/puncture size and result in less bleeding.

For some applications, hemostatic tissue anchor 120 further comprises a flexible elongate tension member 146 coupled to a portion of anchor portion 130. Through flexible elongate tension member 146, or components equivalent thereto, the tensile force can be applied to anchor portion 130 after it has been expanded. When applied in vivo, the tensile force may have the benefit of bringing the anchor close to cardiac tissue wall 160 to which it is applied. For some applications, an anchor system 150 is provided that comprises hemostatic tissue anchor 120 and a tether 152 affixed to flexible elongate tension member 146 such that the tensile force can be applied to hemostatic tissue anchor 120 via tether 152 and flexible elongate tension member 146. Optionally, hemostatic tissue anchor 120 further comprises a tube 154 that surrounds a proximal portion of flexible elongate tension member 146. For some applications, anchor system 150 further comprises a second tissue anchor, separate and distinct from hemostatic tissue anchor 120, such as is shown in above-mentioned PCT Publication WO 2016/087934. For some applications, the second tissue anchor, and additional anchors if so desired, is couplable or coupled to hemostatic tissue anchor 120 by one or more tethers that include tether 152.

Flexible elongate tension member 146 extends through a portion of (a) anchor portion 130 of hemostatic tissue anchor 120 and (b) a distal opening 194 of a passage through hemostatic tissue anchor 120, such that expanded anchor portion 130 can be drawn tightly against the second side of cardiac tissue wall 160 at the target site when the tensile force is applied to anchor portion 130.

Distal opening 194 of the passage is typically located near (e.g., at) a distal end 192 of anchor head 196. A portion of flexible elongate tension member 146 is slidably disposed through the passage. For some applications, the passage is defined by anchor head 196 (as shown). Anchor head 196 may optionally implement techniques described in above-mentioned PCT Publication WO 2016/087934. For some applications, in addition to or instead of elongate anchor shaft 132, anchor head 196 comprises one or more collars 197, such as distal and proximal collars 197A and 197B, as shown, or exactly one collar 197 (configuration not shown). For some of these applications, distal opening 194 is defined by a distal end of distal collar 197A (as shown in FIGS. 1A-B and 2) or a distal end of the exactly one collar 197 (configuration not shown). The passage is typically a channel, but may also be a groove (e.g., a U-shaped groove).

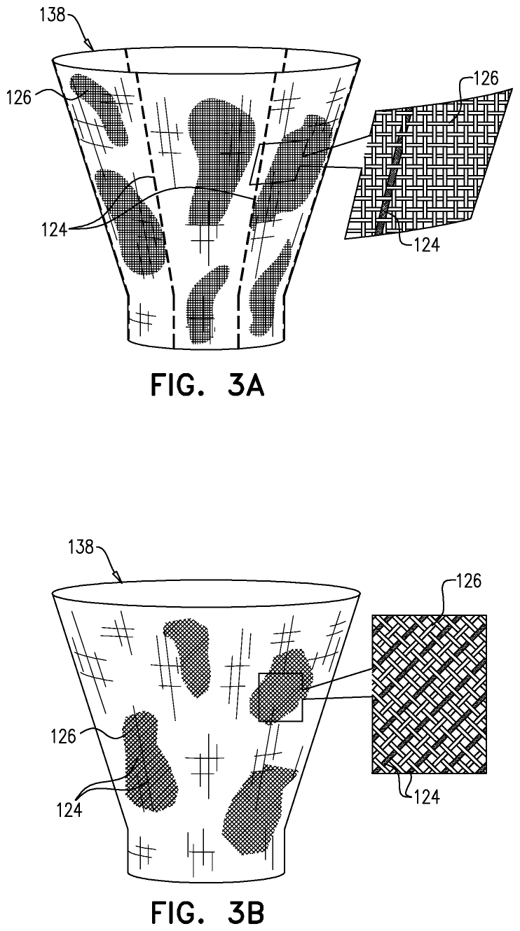

Reference is now made to FIGS. 3A-B, which are schematic illustrations of expanded frustoconical configuration 138 of hemostatic sealing element 122, in accordance with respective applications of the present invention. For some applications, sealing membrane 126 comprises a polymer, which is optionally electrospun. For example, the polymer may comprise PTFE, TPU, HDPE, nylon, PEEK, and/or a hydrogel. Alternatively or additionally, sealing membrane 126 comprises a biocompatible or a bioabsorbable material, which is not necessarily a polymer. For some applications, self-expanding frame 124 is embedded in sealing membrane 126. Alternatively or additionally, for some applications, sealing membrane 126 is dip-coated or laminated onto self-expanding frame 124.

For some applications, such as shown in FIG. 3A, sealing membrane 126 is woven, such as into a mesh. For some applications, sealing membrane 126 comprises a fabric. For some applications, sealing membrane 126 comprises woven Nitinol fibres, e.g., with spacing of less than 6 um (which is the typical size of a blood platelet).

Optionally, in applications in which self-expanding frame 124 comprises metal, the self-expanding frame comprises metal wires integrated into a woven synthetic mesh. For some applications, such as shown in FIG. 3B, self-expanding metal frame 124 comprises metal wires braided into sealing membrane 126.

For some applications of the present invention, hemostatic sealing element 122 is coated with a therapeutic agent. For applications in which hemostatic sealing element 122 is configured to elute a therapeutic agent or is coated with a therapeutic agent, the therapeutic agent may comprise, for example, a fibrosis-enhancing drug, an agent which promotes tissue growth, a clotting agent, an anti-inflammatory, and/or an antibiotic.

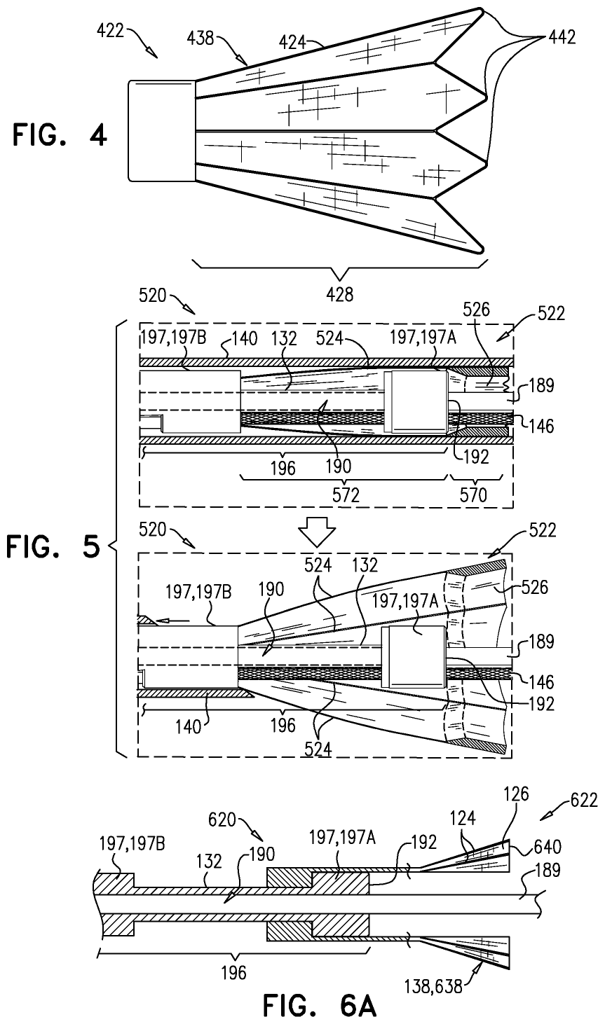

Reference is now made to FIG. 4, which is a schematic illustration of a hemostatic sealing element 422, in accordance with an application of the present invention. Hemostatic sealing element 422 is an alternative configuration of hemostatic sealing element 122, and may be used for both the configurations shown in FIGS. 1A and 1B. In this configuration, hemostatic sealing element 422 comprises a self-expanding frame 424. When an expandable portion 428 of hemostatic sealing element 422 assumes an expanded frustoconical configuration 438, self-expanding frame 424 is shaped so as define a plurality of distally- or proximally-extending crowns 442, which may help ensure radial opposition of hemostatic sealing element 422 to tissue, in order to form a good seal (crowns are shown extending distally in FIG. 4).

Reference is now made to FIG. 5, which is a schematic illustration of a portion of a hemostatic tissue anchor 520, in accordance with an application of the present invention. Other than as described below and shown in FIG. 5, hemostatic tissue anchor 520 is identical to hemostatic tissue anchor 320, described hereinabove with reference to FIG. 1B. A sealing membrane 526 of a hemostatic sealing element 522 has variable thickness. For example, the thickness of sealing membrane 526 may be greater at a first axial location 570 at which sealing membrane 526 axially overlaps wire 189 of anchor portion 130 distal to distal end 192 of anchor head 196 than at a second axial location 572 at which sealing membrane 526 axially overlaps anchor head 196 (which is wider than wire 189), when hemostatic tissue anchor 220 is constrained within hollow delivery shaft 140.

Reference is now made to FIGS. 6A-B, which are schematic illustrations of a hemostatic tissue anchor 620, in accordance with an application of the present invention. FIG. 6A shows a portion of hemostatic tissue anchor 620, and FIG. 6B shows hemostatic tissue anchor 620 anchored to cardiac tissue wall 160. Other than as described below and shown in FIGS. 6A-B, hemostatic tissue anchor 620 is identical to hemostatic tissue anchor 320, described hereinabove with reference to FIG. 1B. Hemostatic tissue anchor 620 comprises a hemostatic sealing element 622, which has an expanded frustoconical configuration 638 that has a distal end 640 that is disposed distal to distal end 192 of anchor head 196 (and thus distal to distal collar 197A in configurations in which distal collar 197A is provided). Distal end 340 may touch, or come near to, anchor portion 130.

As shown in FIG. 6B, in these applications, hemostatic sealing element 322 is typically configured to be disposed only partially within cardiac tissue wall 160 at the target site, with a distal portion of hemostatic sealing element 622, including distal end 640, expanded on the far side of cardiac tissue wall 160, e.g., in the pericardial cavity 180. Expanded frustoconical configuration 638 is partially disposed in cardiac tissue wall 160, and the outer surface of cardiac tissue wall 160 is engaged by the proximal underside of expanded frustoconical configuration 638.

Distal end 192 of anchor head 196 is typically disposed several millimeters proximal to expanded frustoconical configuration 638, so expanded frustoconical configuration 638 begins to taper or flare out distal to distal end 192 of anchor head 196 within cardiac tissue wall 160. Expanded frustoconical configuration 638 thus may be trumpet-bell-shaped. (As used in the present application, including in the claims, the term “frustoconical” includes within its scope shapes that include a strictly conical distal portion, shapes that include a trumpet-bell-shaped distal portion, and shapes that include other similarly-shaped distal portions.) The trumpet-bell shape may optionally flare into a disc-shaped portion 642 near distal end 640 of (i.e., near the distal perimeter of) expanded frustoconical configuration 638, as shown in FIG. 6B.

For some applications, self-expanding frame 124 and sealing membrane 126 are shaped and configured to allow expanded frustoconical configuration 638 to assume the trumpet-bell shape. For some applications, disposition of the distal portion of hemostatic sealing element 622 in the pericardial cavity 180 causes expanded frustoconical configuration 638 to assume the trumpet-bell shape; alternatively or additionally, a shape memory of self-expanding frame 124 and/or sealing membrane 126 cause or contribute to the assumption of the trumpet-bell shape.

Alternatively, expanded frustoconical configuration 638 is configured to main a strictly conical distal portion when expanded in the pericardial cavity 180, similar to the shape of expanded frustoconical configuration 338 shown in FIG. 1B.

For some applications, techniques and apparatus described in one or more of the following applications and/or patents, which are assigned to the assignee of the present application and are incorporated herein by reference, are combined with techniques and apparatus described herein: U.S. Pat. No. 8,475,525 to Maisano et al.; U.S. Pat. No. 8,961,596 to Maisano et al.; U.S. Pat. No. 8,961,594 to Maisano et al.; PCT Publication WO 2011/089601; U.S. Pat. No. 9,241,702 to Maisano et al.; U.S. Provisional Application 61/750,427, filed Jan. 9, 2013; U.S. Provisional Application 61/783,224, filed Mar. 14, 2013; U.S. Provisional Application 61/897,491, filed Oct. 30, 2013; U.S. Provisional Application 61/897,509, filed Oct. 30, 2013; U.S. Pat. No. 9,307,980 to Gilmore et al.; PCT Publication WO 2014/108903; PCT Publication WO 2014/141239; U.S. Provisional Application 62/014,397, filed Jun. 19, 2014; PCT Publication WO 2015/063580; US Patent Application Publication 2015/0119936; U.S. Provisional Application 62/086,269, filed Dec. 2, 2014; U.S. Provisional Application 62/131,636, filed Mar. 11, 2015; U.S. Provisional Application 62/167,660, filed May 28, 2015; PCT Publication WO 2015/193728; PCT Publication WO 2016/087934; US Patent Application Publication 2016/0235533; US Patent Application Publication 2016/0242762; PCT Publication WO 2016/189391; US Patent Application Publication 2016/0262741; U.S. Provisional Application 62/376,685, filed Aug. 18, 2016; U.S. Provisional Application 62/456,206, filed Feb. 8, 2017; U.S. Provisional Application 62/456,202, filed Feb. 8, 2017; U.S. Provisional Application 62/465,410, filed Mar. 1, 2017; U.S. Provisional Application 62/465,400, filed Mar. 1, 2017; PCT Publication WO 2018/035378; U.S. Provisional Application 62/579,281, filed Oct. 31, 2017; U.S. Provisional Application 62/516,894, filed Jun. 8, 2017; U.S. Provisional Application 62/530,372, filed Jul. 10, 2017; and U.S. Provisional Application 62/570,226, filed Oct. 10, 2017.

Patents and patent application publications incorporated by reference in the present patent application are to be considered an integral part of the application except that to the extent any terms are defined in these incorporated patents and patent application publications in a manner that conflicts with the definitions made explicitly or implicitly in the present specification, only the definitions in the present specification should be considered.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
a hollow delivery shaft (140) comprising a hollow needle having a sharp distal end; and
a hemostatic tissue anchor (120) disposed within the hollow needle of the hollow delivery shaft (140), with the sharp distal end of the hollow needle extending distally beyond a distal end of the hemostatic tissue anchor (120), for delivery to a target site, the hemostatic tissue anchor (120) configured to be anchored to a myocardial tissue wall at the target site, the hemostatic tissue anchor (120) comprising:
an anchor portion (130) supported at a distal end (192) of a generally elongate anchor shaft (132), wherein the hollow needle is configured to deliver the anchor portion (130) through the myocardial tissue wall and into the pericardial cavity between visceral pericardium and parietal pericardium, wherein the anchor portion (130) configured to expand from a first generally elongate configuration within the hollow delivery shaft (140) during delivery of the hemostatic tissue anchor (120), to a second expanded configuration, upon release from the hollow delivery shaft (140), such that the anchor portion (130) in the second expanded configuration defines a generally planar structure orthogonal to the elongate anchor shaft (132) that can be drawn tightly against the myocardial tissue wall at the target site when a tensile force is applied to the anchor portion (130); and
a hemostatic sealing element (122), which (a) is coupled to and surrounds at least an axial portion of the elongate anchor shaft (132), and (b) is configured to be disposed at least partially within the myocardial tissue wall at the target site, characterized in that:
the hemostatic sealing element (122) comprises a self-expanding frame (124) attached to a sealing membrane (126),
the hemostatic sealing element (122) comprises an expandable portion (128) that assumes a collapsed configuration (136) within the hollow needle of the hollow delivery shaft (140) during delivery of the hemostatic tissue anchor (120), and, upon release from the hollow needle of the hollow delivery shaft (140) at least partially within the myocardial tissue wall, an expanded frustoconical configuration (138), the expanded frustoconical configuration (138) defined by the self-expanding frame (124) and the sealing membrane (126), and
once the expandable portion (128) of the hemostatic sealing element (122) is implanted at least partially within the myocardial tissue wall at the target site, the expanded frustoconical configuration (138) of the hemostatic sealing element (122) acts as a hemostatic seal of an opening through the myocardial tissue wall, through which opening the elongate anchor shaft (132) is disposed.

2. The apparatus according to claim 1, wherein the expanded frustoconical configuration (138) widens in a distal direction.

3. The apparatus according to claim 1, wherein the expanded frustoconical configuration (138) widens in a proximal direction.

4. The apparatus according to claim 1, wherein the self-expanding frame (124) is embedded in the sealing membrane (126).

5. The apparatus according to claim 1, wherein the sealing membrane (126) is electrospun.

6. The apparatus according to claim 1, wherein the sealing membrane (126) is dip-coated or laminated onto the self-expanding frame (124).

7. The apparatus according to claim 1, wherein the sealing membrane (126) is woven.

8. The apparatus according to claim 1, wherein the sealing membrane (126) comprises a fabric.

9. The apparatus according to claim 1, wherein the sealing membrane (126) comprises a hygroscopic polymer, which, when exposed to fluid, absorbs moisture and expands.

10. The apparatus according to claim 1, wherein the self-expanding frame (124) of the expanded frustoconical configuration (138) is shaped so as define a plurality of distally- or proximally-extending crowns.

11. The apparatus according to claim 1, wherein the self-expanding frame (124) comprises metal.

12. The apparatus according to claim 11, wherein the self-expanding metal frame (124) comprises metal wires braided into the sealing membrane (126).

13. The apparatus according to claim 1, wherein the self-expanding frame (124) comprises a hygroscopic polymer, which, when exposed to fluid, absorbs moisture and expands, thereby driving the expandable portion (128) to assume the expanded frustoconical configuration (138).

14. The apparatus according to claim 1, wherein the expanded frustoconical configuration (138) has a greatest diameter that is greater than an outer diameter of the hollow delivery shaft (140).

15. The apparatus according to claim 1, wherein the elongate anchor shaft (132) comprises an anchor head that defines the distal end (192) of the anchor shaft (132), wherein the expanded frustoconical configuration (138) has a distal end that is disposed proximal to the distal end (192) of the anchor head, and wherein the hemostatic sealing element (122) is configured to be disposed entirely within the myocardial tissue wall at the target site.

16. The apparatus according to claim 1, wherein the elongate anchor shaft (132) comprises an anchor head that defines the distal end (192) of the anchor shaft (132), wherein the expanded frustoconical configuration (138) has a distal end that is disposed distal to the distal end (192) of the anchor head, and wherein the hemostatic sealing element (122) is configured to be disposed only partially within the myocardial tissue wall at the target site, with a distal portion of the hemostatic sealing element (122), including the distal end of the expanded frustoconical configuration (138), expanded in the pericardial cavity between visceral pericardium and parietal pericardium.

17. The apparatus according to claim 16, wherein the hemostatic sealing element (122) is configured such that when the distal portion of the hemostatic sealing element (122) is expanded in the pericardial cavity, the distal portion of the hemostatic sealing element (122) assumes a trumpet-bell shape.

18. The apparatus according to claim 16, wherein the sealing membrane (126) has a greater thickness at a first axial location at which the sealing membrane (126) axially overlaps a wire of the anchor portion (130) distal to the distal end (192) of the anchor head than at a second axial location at which the sealing membrane (126) axially overlaps the anchor head when the hemostatic tissue anchor (120) is constrained within the hollow delivery shaft (140).

19. (canceled)

20. The apparatus according to claim 1, wherein the anchor portion (130) is configured to be implanted in the pericardial cavity between visceral pericardium and parietal pericardium, generally alongside and against the parietal pericardium, without penetrating the parietal pericardium.

21. (canceled)

22. An anchor system (150) comprising:
a hemostatic tissue anchor (120) deliverable within a hollow delivery shaft (140) to a target site, the hemostatic tissue anchor (120) configured to be anchored to a cardiac tissue wall at the target site, the hemostatic tissue anchor (120) comprising:
an anchor portion (130) supported at a distal end (192) of a generally elongate anchor shaft (132), the anchor portion (130) configured to expand from a first generally elongate configuration within the hollow delivery shaft (140) during delivery of the hemostatic tissue anchor (120), to a second expanded configuration, upon release from the hollow delivery shaft (140), such that the anchor portion (130) in the second expanded configuration defines a generally planar structure orthogonal to the elongate anchor shaft (132) that can be drawn tightly against the cardiac tissue wall at the target site when a tensile force is applied to the anchor portion (130); and
a hemostatic sealing element (122), which (a) is coupled to and surrounds at least an axial portion of the elongate anchor shaft (132), (b) is configured to be disposed at least partially within the cardiac tissue wall at the target site;
a second tissue anchor, separate and distinct from the hemostatic tissue anchor (120); and
a tether (152) that couples the second tissue anchor to the hemostatic tissue anchor (120), characterized in that:
the hemostatic sealing element (122) comprises a self-expanding frame (124) attached to a sealing membrane (126),
the hemostatic sealing element (122) comprises an expandable portion (128) that assumes a collapsed configuration (136) within the hollow delivery shaft (140) during delivery of the hemostatic tissue anchor (120), and, upon release from the hollow delivery shaft (140) at least partially within the cardiac tissue wall, an expanded frustoconical configuration (138), the expanded frustoconical configuration (138) defined by the self-expanding frame (124) and the sealing membrane (126), and
once the expandable portion (128) of the hemostatic sealing element (122) is implanted at least partially within the cardiac tissue wall at the target site, the expanded frustoconical configuration (138) of the hemostatic sealing element (122) acts as a hemostatic seal of an opening through the cardiac tissue wall, through which opening the elongate anchor shaft (132) is disposed.

23. The anchor system according to claim 22, wherein the expanded frustoconical configuration (138) widens in a distal direction.

24. The anchor system according to claim 22, wherein the expanded frustoconical configuration (138) widens in a proximal direction.

25. The anchor system according to claim 22, wherein the self-expanding frame (124) is embedded in the sealing membrane (126).

26. The anchor system according to claim 22, wherein the sealing membrane (126) is electrospun.

27. The anchor system according to claim 22, wherein the sealing membrane (126) is dip-coated or laminated onto the self-expanding frame (124).

28. The anchor system according to claim 22, wherein the sealing membrane (126) is woven.

29. The anchor system according to claim 22, wherein the sealing membrane (126) comprises a fabric.

30. The anchor system according to claim 22, wherein the sealing membrane (126) comprises a hygroscopic polymer, which, when exposed to fluid, absorbs moisture and expands.

31. The anchor system according to claim 22, wherein the self-expanding frame (124) of the expanded frustoconical configuration (138) is shaped so as define a plurality of distally- or proximally-extending crowns.

32. The anchor system according to claim 22, wherein the self-expanding frame (124) comprises metal.

33. The anchor system according to claim 32, wherein the self-expanding metal frame (124) comprises metal wires braided into the sealing membrane (126).

34. The anchor system according to claim 22, wherein the self-expanding frame (124) comprises a hygroscopic polymer, which, when exposed to fluid, absorbs moisture and expands, thereby driving the expandable portion (128) to assume the expanded frustoconical configuration (138).

35. The anchor system according to claim 22, wherein the expanded frustoconical configuration (138) has a greatest diameter that is greater than an outer diameter of the hollow delivery shaft (140).

36. The anchor system according to claim 22, wherein the elongate anchor shaft (132) comprises an anchor head that defines the distal end (192) of the anchor shaft (132), wherein the expanded frustoconical configuration (138) has a distal end that is disposed proximal to the distal end (192) of the anchor head, and wherein the hemostatic sealing element (122) is configured to be disposed entirely within the cardiac tissue wall at the target site.

37. The anchor system according to claim 22, wherein the elongate anchor shaft (132) comprises an anchor head that defines the distal end (192) of the anchor shaft (132), wherein the expanded frustoconical configuration (138) has a distal end that is disposed distal to the distal end (192) of the anchor head, and wherein the hemostatic sealing element (122) is configured to be disposed only partially within the cardiac tissue wall at the target site, with a distal portion of the hemostatic sealing element (122), including the distal end of the expanded frustoconical configuration (138), expanded in the pericardial cavity between visceral pericardium and parietal pericardium.

38. The anchor system according to claim 37, wherein the hemostatic sealing element (122) is configured such that when the distal portion of the hemostatic sealing element (122) is expanded in the pericardial cavity, the distal portion of the hemostatic sealing element (122) assumes a trumpet-bell shape.

39. The anchor system according to claim 37, wherein the sealing membrane (126) has a greater thickness at a first axial location at which the sealing membrane (126) axially overlaps a wire of the anchor portion (130) distal to the distal end (192) of the anchor head than at a second axial location at which the sealing membrane (126) axially overlaps the anchor head when the hemostatic tissue anchor (120) is constrained within the hollow delivery shaft (140).

40. The anchor system according to claim 22, wherein the cardiac tissue wall is a myocardial tissue wall, and wherein the expandable portion (128) of the hemostatic sealing element (122) is configured to be implanted at least partially within the myocardial tissue wall.

41. The anchor system according to claim 40, wherein the anchor portion (130) is configured to be implanted in the pericardial cavity between visceral pericardium and parietal pericardium, generally alongside and against the parietal pericardium, without penetrating the parietal pericardium.

42. The anchor system according to claim 22,
wherein the hemostatic tissue anchor (120) further comprises a flexible elongate tension member (146) coupled to a portion of the anchor portion (130), and
wherein the tether (152) is affixed to the flexible elongate tension member (146) such that the tensile force can be applied to hemostatic tissue anchor (120) via the tether (152) and the flexible elongate tension member (146).
